(19) United States
(12) Patent Application Publication
(10) Pub. No.: US 2002/0042514 A1
(43) Pub. Date: Apr. 11, 2002

(54) Synthesis of chlorinated pyrimidines

(76) Inventors: Timothy John Doyle, Bucks, AL (US); Peter Karl Wehrenberg, Bucks, AL (US); Michael Charles Henry Standen, Bucks, AL (US)

Correspondence Address: Edward D. Grieff, Esq., Hale and Dorr LLP, 1455 Pennsylvania Avenue, NW, Washington, DC, 20004, US

(21) Appl. No.: 09/875,934

(22) Filed: Jun. 8, 2001

Related U.S. Application Data
(60) Provisional application No. 60/214,121, filed on Jun. 26, 2000.

(51) Int. Cl. C07D 239/02

(52) U.S. Cl. 544/334

(57) ABSTRACT

A facile process for the preparation of 4,6-dichloropyrimidine is provided, which utilizes quaternary ammonium salts or quaternary phosphonium salts as catalysts for the reaction of, for example, 4,6-dihydroxypyrimidine or 4-chloro-6-methoxypyrimidine with phosgene.

### FIELD OF THE INVENTION

This invention belongs to the field of organic chemistry. In particular, it relates to a process for preparing 4,6-dichloropyrimidine via the reaction of phosgene with, for example, 4,6-dihydroxypyrimidine in the presence of a quaternary ammonium salt or quaternary phosphonium salt.

### BACKGROUND OF THE INVENTION

4,6-Dichloropyrimidine (DCP) is useful as an intermediate in the preparation of certain agricultural products. For example, U.S. Pat. No. 5,145,856 and WO92/08703A1 describe certain compounds useful as fungicides, which utilize DCP as a key synthetic intermediate.

U.S. Pat. No. 5,723,612 describes the preparation of DCP via the reaction of 4,6-dihydroxypyrimidine with phosphorous oxychloride in the presence of a stoichiometric amount of a trialkylamine as acid scavenger and catalyst.

U.S. Pat. No. 6,018,045 describes a process for preparing DCP via the treatment of 4,6-dihydroxypyrimidine with phosphorous oxychloride in the presence of a secondary or tertiary saturated amine, the hydrochloride salt of a secondary or tertiary saturated hindered amine, or an unsaturated 5-membered tertiary nitrogen-containing ring. The basic amines act as acid scavengers.

Research Disclosure 39104 (November 1996) describes the preparation of DCP via the chlorination of 4,6-dihydroxypyrimidine or 4-chloro-6-methoxypyrimidine. In this reaction, the chlorinating agent is a compound of the formula R₃PCl₂, wherein R is a phenyl group or an alkyl group, or one of the R groups is linked to a polymer support.

A process for preparing 4,6-dichloropyrimidine from 4,6-dihydroxypyrimidine using phosgene in the presence of a suitable acid scavenger is described in WO95/29166(U.S. Pat. No. 5,750,694). Suitable bases include tertiary amines and heterocyclic amines. A process for preparing 2-chloromethyl-4,5,6-trichloropyrimidine from 5,5-dichloro-4,5-dihydro-6-hydroxy-2-trichloromethylpyrimidine 4-one using a chlorinating agent in the presence of a catalyst is described in U.S. Pat. No. 4,668,788. The catalysts employed are generally either acid scavengers (amines) or activate the chlorinating agent's Cl—X bond (I₂, sulfur, Friedel-Crafts catalysts, the various phosphorous compounds mentioned, etc.), where X=—Cl, —SO₂Cl, —NR₁R₂, —I, —Br, —PCl₄ by either coordinating with X, as in the case of Friedel-Crafts catalysts, or forming a covalent bond, e.g., ICl. In Example 7, phosgene is used as the chlorinating agent and triphenylphosphine oxide as catalyst. A similar process is described in EP-A-0095637 for the chlorination of 2,3-dihydroxyquinoxalin-6-carboxylic acid.

None of these documents suggest the use of quaternary ammonium and quaternary phosphonium salts as set forth in the present invention. Such salts are not capable of acting as acid scavengers or activating the chlorination agent's Cl—X bond through either coordination with X or formation of a covalent bond. Thus, the efficacy of these salts was unexpected.

### SUMMARY OF THE INVENTION

The present invention provides a facile process for the preparation of 4,6-dichloropyrimidine, which utilizes quaternary ammonium salts or quaternary phosphonium salts as catalysts in the reaction of certain hydroxy, halo, and alkoxy substituted pyrimidines with phosgene. In a preferred embodiment, 4,6-dihydroxypyrimidine or 4-chloro-6-methoxypyrimidine is reacted with phosgene in the presence of tricaprylylmethylammonium chloride or tributylmethylammonium chloride.

### DETAILED DESCRIPTION OF THE INVENTION

The present invention provides a process for preparing 4,6-dichloropyrimidine which comprises contacting a compound of Formula (I)

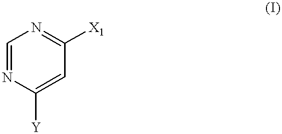

wherein X₁ and Y are independently selected from hydroxy, C₁- C₄ alkoxy and halo, with phosgene, in the presence of at least one quaternary ammonium salt or quaternary phosphonium salt.

In the process of the present invention it is preferred that the starting material of Formula (I) is either 4,6-dihydroxypyrimidine or 4-chloro-6-methoxypyrimidine.

In the above process, it will be understood that all references to 4,6-dihydroxy pyrimidine include its tautomeric forms, i.e.,

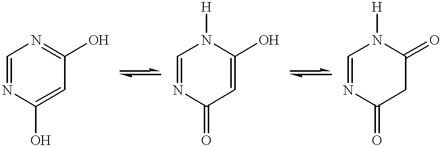

The quaternary ammonium salts and quaternary phosphonium salts are known compounds and can either be prepared using methodologies well-known in the art, or are available commercially.

Examples of preferred quaternary ammonium and quaternary phosphonium catalysts include compounds of Formula (II)

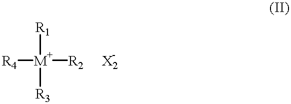

wherein R₁, R₂, R₃, and R₄, are independently selected from branched or linear C₁-C₁₆ alkyl, substituted aryl, benzyl, capryl, phenyl, and trityl;

M is P or N; and

X₂ is halo, hydrogen sulfate, tetrafluoroborate, trifluoromethanesulfonate, acetate, perchlorate, dihydrogenphosphate, hexafluoroantimonate, or nitrate.

Examples of compounds of Formula (II) include those set forth in the table below:

| R₁ | R₂ | R₃ | R₄ | M | X₂ |
|---|---|---|---|---|---|
| methyl | methyl | methyl | methyl | N | Cl |
| ethyl | ethyl | ethyl | ethyl | N | Cl |
| phenyl | methyl | methyl | methyl | N | Cl |
| benzyl | methyl | methyl | methyl | N | Cl |
| n-butyl | n-butyl | n-butyl | methyl | N | Cl |
| benzyl | ethyl | ethyl | ethyl | N | Cl |
| benzyl | n-propyl | n-propyl | n-propyl | N | Cl |
| n-butyl | n-butyl | n-butyl | n-butyl | N | Cl |
| benzyl | n-butyl | n-butyl | n-butyl | N | Cl |
| methyl | capryl | capryl | capryl | N | Cl |
| n-pentyl | n-pentyl | n-pentyl | n-pentyl | N | Cl |
| phenyl | phenyl | phenyl | phenyl | P | Cl |
| trityl | phenyl | phenyl | phenyl | P | Cl |
| n-butyl | n-butyl | n-butyl | n-butyl | P | Cl |
| methyl | methyl | methyl | methyl | N | Br |
| methyl | methyl | methyl | methyl | N | I |
| methyl | ethyl | ethyl | ethyl | N | Br |
| ethyl | ethyl | ethyl | ethyl | N | Br |
| ethyl | ethyl | ethyl | ethyl | N | I |
| ethyl | ethyl | ethyl | ethyl | N | acetate |
| ethyl | ethyl | ethyl | ethyl | N | tetrafluoro- |
| | | | | | borate |
| n-hexyl | methyl | methyl | methyl | N | Br |
| phenyl | methyl | methyl | methyl | N | I |
| n-octyl | methyl | methyl | methyl | N | Br |
| benzyl | methyl | methyl | methyl | N | F |
| benzyl | methyl | methyl | methyl | N | Br |
| benzyl | methyl | methyl | methyl | N | I |
| n-nonyl | methyl | methyl | methyl | N | Br |
| n-propyl | n-propyl | n-propyl | n-propyl | N | Br |
| n-propyl | n-propyl | n-propyl | n-propyl | N | hydrogen |
| | | | | | sulfate |
| n-propyl | n-propyl | n-propyl | n-propyl | N | trifluoro- |
| | | | | | methane- |
| | | | | | sulfonate |
| phenyl | ethyl | ethyl | ethyl | N | I |
| n-decyl | methyl | methyl | methyl | N | Br |
| benzyl | ethyl | ethyl | ethyl | N | tetrafluoro- |
| | | | | | borate |
| benzyl | ethyl | ethyl | ethyl | N | Br |
| n-dodecyl | methyl | methyl | methyl | N | Cl |
| n-dodecyl | methyl | methyl | methyl | N | Br |
| n-butyl | n-butyl | n-butyl | n-butyl | N | Br |
| n-butyl | n-butyl | n-butyl | n-butyl | N | I |
| n-butyl | n-butyl | n-butyl | n-butyl | N | acetate |
| n-butyl | n-butyl | n-butyl | n-butyl | N | hydrogen |
| n-butyl | n-butyl | n-butyl | n-butyl | N | trifluoro |
| | | | | | methane- |
| | | | | | sulfonate |
| n-butyl | n-butyl | n-butyl | n-butyl | N | dihydrogen |
| | | | | | -phosphate |
| n-butyl | n-butyl | n-butyl | n-butyl | N | perchlorate |
| | | | | | (ClO₄) |
| n-pentyl | n-pentyl | n-pentyl | n-pentyl | N | I |
| benzyl | butyl | butyl | butyl | N | Br |
| n-hexyl | n-hexyl | n-hexyl | n-hexyl | N | Br |
| n-hexyl | n-hexyl | n-hexyl | n-hexyl | N | hydrogen |
| | | | | | sulfate |
| n-octyl | n-octyl | n-octyl | n-octyl | N | Br |
| n-dodecyl | n-dodecyl | n-dodecyl | n-dodecyl | N | I |
| n-dodecyl | n-dodecyl | n-dodecyl | n-dodecyl | N | nitrate |
| ethyl | ethyl | ethyl | ethyl | N | F |
| n-butyl | n-butyl | n-butyl | n-butyl | P | Br |
| phenyl | phenyl | phenyl | phenyl | P | Br |
| phenyl | phenyl | phenyl | phenyl | P | I |
| phenyl | phenyl | phenyl | phenyl | P | hexafluoro- |
| | | | | | antimonate |
| phenyl | phenyl | phenyl | phenyl | P | tetrafluoro- |
| | | | | | borate |
| n- | n-butyl | n-butyl | n-butyl | P | Br |
| hexadecyl | | | | | |

Especially preferred catalysts are selected from the following:

benzyltributylammonium chloride; benzyltriphenylphosphonium chloride; tricaprylylmethylammonium chloride; tributylammonium chloride; and tributylmethylammonium chloride.

Examples of quaternary ammonium compounds include ALIQUAT 336 and ALIQUAT 175, available from Cognis Corporation.

In a preferred embodiment, the starting material of Formula (I) is slurried in an aprotic solvent along with the catalyst of Formula (II) and heated to a temperature of up to 160°, preferably about 90° C. to 160° C., more preferably about 100° C. to 110° C., most preferably about 105° to 110° C., and treated with phosgene.

In this preferred embodiment, examples of suitable solvents include butyronitrile, nitrobenzene, benzonitrile, o-tolunitrile, m-tolunitrile, acetonitrile, o-xylene, and proprionitrile. Alternatively, the solvent may be less polar solvents or the end product, 4,6-dichloropyrimidine can be utilized as the solvent.

When the compound of Formula (I) is 4,6-dihydroxypyrimidine, preferred solvents include m-tolunitrile, o-tolunitrile, and nitrobenzene. Further, in such a case it is preferred that the quaternary ammonium salt or quaternary phosphonium salt catalyst be present, relative to the starting material of Formula (I), in a molar ratio of about 1:100 to 1:5, especially 1:5- to 1:20. The amount of phosgene used is preferably from about 2.5 to 4 molar equivalents.

When the compound of Formula (I) is 4-chloro-6-methoxypyrimidine, preferred solvents include o-xylene and acetonitrile; alternatively, the reaction can be run without solvent, i.e., in neat 4-chloro-6-methoxypyrimidine. Further, in such a case, it is preferred that the quaternary ammonium salt or quaternary phosphonium salt catalyst be present, relative to the starting material of Formula (I), in a molar ratio of about 1:20 to 1:1, especially 1:20 to 1:1.5. The amount of phosgene used is preferably from about 1.1 to 2.2 molar equivalents.

As noted above, DCP is useful as an intermediate in the preparation of certain agricultural products. For example, U.S. Pat. No. 5,145,856, incorporated herein by reference, and WO92/08703A1 describe certain compounds useful as fungicides, which utilize DCP as a key synthetic intermediate. Moreover, the commercial product known as Azoxystrobin can be manufactured using DCP as a key intermediate as per the following reaction scheme:

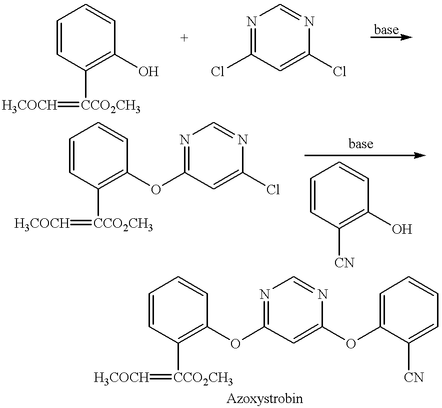

In the above reaction scheme, bases such as potassium carbonate or sodium methoxide may be utilized. In an alternative embodiment, the starting material may be a lactone having the following structure:

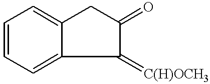

In such a case, a metal salt of a C₁-C₆ alkoxide such as sodium methoxide should be utilized (see WO92/08703 A1).

Accordingly, in a further aspect of the present invention, there is provided the process of the invention as set forth herein, further comprising the steps of contacting DCP with at least one compound of the formula

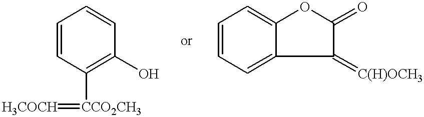

in the presence of a base to afford an intermediate of the formula

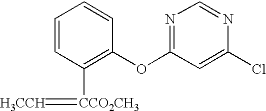

followed by contacting said intermediate with 2-cyanophenol in the presence of a base to afford Azoxystrobin.

### EXPERIMENTAL SECTION

### EXAMPLES 1-6

General Procedure for Conversion of 4,6-dihydroxypyrimidine (DHP) to 4,6-dichloropyrimidine (DCP): The reaction vessel is a Morton-type flask fitted with a heating mantle, a mechanical agitator, a temperature probe, a phosgene dip pipe (which also serves as a nitrogen inlet when phosgene is not being introduced to the reactor), and a dry ice condenser. The dry ice condenser is vented into a caustic scrubber. The reactor is charged with 4,6-dihydroxypyrimidine, solvent, and catalyst, forming a slurry. The agitator is started and the mixture is heated to 105°-110° C. When this temperature range is reached, phosgene gas is introduced subsurface to the reaction mixture via the dip pipe. Phosgene addition is continued over 3-5 hours. During the addition, phosgene escaping the reaction is condensed by the dry ice condenser and returned to the reactor. This reflux of phosgene begins shortly after the phosgene addition is begun, and continues throughout the course of the reaction. After the full charge of phosgene has been added, a post reaction of approximately one hour is usually required to bring the reaction to completion. During this time, the reaction mixture continues to stir at the reaction temperature. The progress of the reaction is followed by monitoring the disappearance of DHP using liquid chromatography. The reaction yield is assessed by liquid chromatographic analysis of the reaction mixture.

TABLE 1
Synthesis of 4,6-Dichloropyrimidine from 4,6-Dihydroxypyrimidine

| | DIP | | Solvent | | Catalyst | Phosgene | |
|---|---|---|---|---|---|---|---|
| | Charge | | Charge | | Charge | Charge | DCP |
| Example | (moles) | Solvent | (moles) | Catalyst | (moles) | (moles) | Yield |
| 1 | 0.05 | butyronitrile | 1.14 | BTBAC | 0.01 | 0.21 | 91% |
| 2 | 0.13 | butyronitrile | 3.41 | BTPPC | 0.03 | 0.53 | 79% |
| 3 | 1.90 | nitrobenzene | 9.49 | TCMAC | 0.09 | 5.46 | 94% |
| 4 | 1.90 | benzonitrile | 11.33 | TCMAC | 0.09 | 5.18 | 88% |
| 5 | 1.55 | o-tolunitrile | 8.11 | TCMAC | 0.08 | 4.59 | 94% |
| 6 | 1.10 | m-tolunitrile | 2.96 | TCMAC | 0.06 | 3.10 | 93% |

Note:
DHP = 4,6-dihydroxypyrimidine
DCP = 4,6-dichloropyrimidine
BTBAC = benzyltributylammonium chloride
BTPPC = benzyltriphenylphosphonium chloride
TCMAC = tricaprylylmethylammonium chloride (ALIQUAT 336)

### EXAMPLES 7-12

General Procedure for Conversion of 4-chloro-6-methoxypyrimidine (CMP) to 4,6-dichloropyrimidine (DCP): The reaction vessel is a Morton-type flask fitted with a heating mantle, a mechanical agitator, a temperature probe, a phosgene dip pipe (which also serves as a nitrogen inlet when phosgene is not being introduced to the reactor), and a dry ice condenser. The dry ice condenser is vented into a caustic scrubber. The reactor is charged with CMP, solvent, and catalyst. The agitator is started and the mixture is heated to 100°-110° C. When this temperature range is reached, phosgene gas is introduced subsurface to the reaction mixture via the dip pipe. Phosgene addition is continued over 3-5 hours. During the addition, phosgene escaping the reaction mixture is condensed by the dry ice condenser and returned to the reaction mixture. This reflux of phosgene begins shortly after the phosgene addition is begun, and continues throughout the course of the reaction. After the full charge of phosgene has been added a post reaction of one hour is usually required to bring the reaction to completion. During this time, the reaction mixture continues to stir at the reaction temperature. The progress of the reaction is followed by monitoring the disappearance of CMP using liquid chromoatography (and/or gas chromatography). The reaction yield is assessed by liquid chromatographic analysis of the reaction mixture.

TABLE 2
Synthesis of 4,6-Dichloropyrimidine from 4-Chloro-6-methoxypyrimidine

| | CMP | | Solvent | | Catalyst | Phosgene | |
|---|---|---|---|---|---|---|---|
| | Charge | | Charge | | Charge | Charge | DCP |
| Example | (moles) | Solvent | (moles) | Catalyst | (moles) | (moles) | Yield |
| 7 | 2.47 | acetonitrile | 1.536 | TCMAC | 0.12 | 3.26 | 97% |
| 8 | 0.64 | acetonitrile | 0.48 | TMAC | 0.06 | 1.20 | 80% |
| 9 | 0.64 | acetonitrile | 0.29 | TBMAC | 0.03 | 0.80 | 97% |
| 10 | 0.52 | o-xylene | 0.93 | TCMAC | 0.37 | 1.12 | 95% |
| 12 | 0.52 | o-xylene | 1.65 | TCMAC | 0.19 | 1.02 | 98% |

Note:
CMP = 4-chloro-6-methoxypyrimidine
DCP = 4,6-dichloropyrimidine
TCMAC = tricaprylylmethylammonium chloride (ALIQUAT 336)
TMAC = tetramethylammonium chloride
TBMAC = tributylmethylammonium chloride (ALIQUAT 175)

1. A process for preparing 4,6-dichloropyrimidine which comprises contacting a compound of Formula (I) 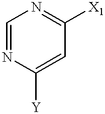
wherein X₁ and Y are independently selected from hydroxy, C₁-C₄ alkoxy and halo; with phosgene, in the presence of at least one quaternary ammonium salt or quaternary phosphonium salt.

2. The process of claim 1, wherein the quaternary ammonium salt and quaternary phosphonium salt is a compound of Formula (II): 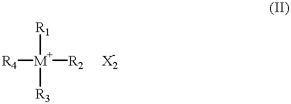
wherein R₁, R₂, R₃, and R₄, are independently selected from branched or linear
C₁-C₁₆ alkyl, substituted aryl, benzyl, capryl, phenyl, and trityl;
M is P or N; and
X₂ is halo, hydrogen sulfate, tetrafluoroborate, trifluoromethanesulfonate, acetate, perchlorate, dihydrogenphosphate, hexafluoroantimonate, or nitrate:

3. The process of claim 1, wherein X₁ and Y are hydroxy.

4. The process of claim 1, wherein X₁ is C₁-C₄ alkoxy and Y is halo.

5. The process of claim 1, wherein X₁ is methoxy and Y is chloro.

6. The process of claim 2, wherein the compound of Formula (II) is selected from the group consisting of benzyltributylammonium chloride, benzyltributylammonium bromide, benzyltriphenylphosphonium chloride, tricaprylylmethylammonium chloride, tributylammonium chloride, and tributylmethylammonium chloride.

7. The process of claim 6, wherein X₁ and Y are hydroxy.

8. The process of claim 6, wherein X₁ is methoxy and Y is chloro.

9. The process of claim 1, further comprising the steps of contacting 4,6-dichloropyrimidine with at least one compound of the formula 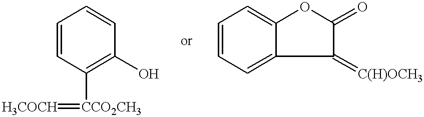
in the presence of a base to afford an intermediate of the formula 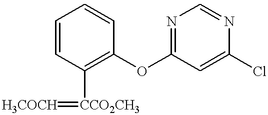
followed by contacting said intermediate with 2-cyanophenol in the presence of a base to afford a compound of the formula

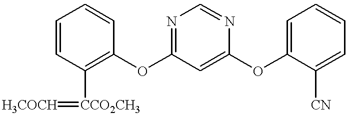

10. The process of claim 9, wherein 4,6-dichloropyrimidine is contacted with the compound of the formula 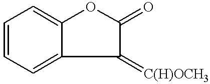
in the presence of a metal salt of a C₁-C₆ alkoxide to afford an intermediate of the formula 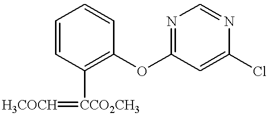
followed by contacting said intermediate with 2-cyanophenol in the presence of a base to afford a compound of the formula

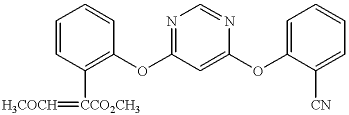

11. The process of claim 10, wherein the metal salt of a C₁-C₆ alkoxide is sodium methoxide.
